(19) United States
(12) Patent Application Publication
(10) Pub. No.: US 2026/0156739 A1
(43) Pub. Date: Jun. 4, 2026

(54) DEVICES INCLUDING ACCESS TO AN ELECTRICALLY CONDUCTIVE COMPONENT AND METHODS OF MAKING AND USING SAME

(71) Applicant: Solventum Intellectual Properties Company, Maplewood, MN (US)

(72) Inventors: Kevin T. REDDY, Maplewood, MN (US); Mikhail L. PEKUROVSKY, Maplewood, MN (US); Kayla C. NICCUM, Maplewood, MN (US); Kara A. MEYERS, Maplewood, MN (US); Shawn C. DODDS, Maplewood, MN (US); Saagar A. SHAH, Maplewood, MN (US)

(73) Assignee: Solventum Intellectual Properties Company, Maplewood, MN (US)

(21) Appl. No.: 19/124,143

(22) PCT Filed: Oct. 17, 2023
(86) PCT No.: PCT/IB2023/060471

§ 371 Date: Apr. 24, 2025

Related U.S. Application Data
(60) Provisional application No. 63/421,682, filed on Nov. 2, 2022.

(51) Int. Cl. H05K 1/02; A61B 5/257; A61B 5/28; H05K 3/10; H05K 3/28

(52) U.S. Cl. CPC H05K 1/0277; H05K 3/107; H05K 3/284; A61B 5/257; A61B 5/28

(57) ABSTRACT

The present disclosure provides a device including a flexible substrate having an adhesive surface on a first major side as well as an access component that includes a first electrically conductive component and a masking component that covers the first electrically conductive component opposite the adhesive surface of the flexible substrate. The first electrically conductive component is adhesively bonded to the adhesive surface of the flexible substrate. The device also includes a second electrically conductive component electrically connected to the first electrically conductive component and that extends from the first electrically conductive component on the first major side of the flexible substrate through to the second major side of the flexible substrate. Further, the device includes an encapsulating layer that encapsulates the masking component and at least a portion of the first major side of the flexible substrate. The disclosure also provides a method of making the device, plus a method of using the device. The method of using the device includes removing at least a portion of the masking component from the device to expose the first electrically conductive component at the first major side of the flexible substrate.

### BACKGROUND

Flexible electronic devices have been prepared and further developments in their structures would be beneficial.

### SUMMARY

In a first aspect, a device is provided. The device includes a flexible substrate including an adhesive surface on a first major side thereof and an access component that includes a first electrically conductive component and a masking component that covers the first electrically conductive component opposite the adhesive surface of the flexible substrate. The first electrically conductive component is adhesively bonded to the adhesive surface of the flexible substrate. The device also includes a second electrically conductive component electrically connected to the first electrically conductive component and that extends from the first electrically conductive component on the first major side of the flexible substrate through to the second major side of the flexible substrate. Further, the device includes an encapsulating layer that encapsulates the masking component and at least a portion of the first major side of the flexible substrate.

In a second aspect, a method of making a device is provided. The method includes obtaining a flexible substrate having an adhesive surface on a first major side thereof and an access component that includes a first electrically conductive component and a masking component that covers the first electrically conductive component opposite the adhesive surface of the flexible substrate. The first electrically conductive component is adhesively bonded to the adhesive surface of the flexible substrate. The method also includes providing a second electrically conductive component electrically connected to the first electrically conductive component and that extends from the first electrically conductive component on the first major side of the flexible substrate through to the second major side of the flexible substrate. Further, the method includes applying an encapsulating layer that encapsulates the masking component and at least a portion of the first major side of the flexible substrate.

In a third aspect, a method of using a device is provided. The method includes obtaining a device according to the first aspect and removing at least a portion of the masking component from the device to expose the first electrically conductive component at the first major side of the flexible substrate.

The above summary of the present disclosure is not intended to describe each disclosed embodiment or every implementation of the present disclosure. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE DRAWINGS

FIGS. 1A-1G are schematic cross-sectional views of methods of making and/or using an exemplary device according to the present disclosure.

FIG. 2 is a schematic cross-sectional view of a portion of an exemplary device according to the present disclosure.

FIG. 3 is a schematic cross-sectional view of another exemplary device according to the present disclosure.

FIG. 4 is a schematic cross-sectional view of a further exemplary device according to the present disclosure.

FIG. 5 is a schematic cross-sectional view of yet another exemplary device according to the present disclosure.

FIGS. 6A-6G are photographs of a process of making an exemplary device according to Example 1.

FIG. 6H is a photograph of the exemplary device of Example 1 in use.

FIGS. 7A-7E are photographs of a process of making an exemplary device according to Example 2.

FIG. 7F is a photograph of the exemplary device of Example 2 in use.

While the above-identified figures set forth several embodiments of the disclosure other embodiments are also contemplated, as noted in the description. The figures are not necessarily drawn to scale. In all cases, this disclosure presents the invention by way of representation and not limitation.

### DETAILED DESCRIPTION OF ILLUSTRATIVE EMBODIMENTS

By using terms of orientation such as “atop”, “on”, “over,” “covering”, “uppermost”, “underlying” and the like for the location of various elements in the disclosed devices, we refer to the relative position of an element with respect to a horizontally-disposed, upwardly-facing substrate.

However, unless otherwise indicated, it is not intended that the substrate or devices should have any particular orientation in space during or after manufacture.

As used herein, referring to features as “first”, “second”, “third”, and so on is solely for clarity to distinguish which feature is being described in a particular embodiment.

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the disclosure.

In this application, terms such as “a”, “an”, and “the” are not intended to refer to only a singular entity, but include the general class of which a specific example may be used for illustration. The terms “a”, “an”, and “the” are used interchangeably with the term “at least one.” The phrases “at least one of” and “comprises at least one of” followed by a list refers to any one of the items in the list and any combination of two or more items in the list.

As used herein, the term “or” is generally employed in its usual sense including “and/or” unless the content clearly dictates otherwise. The term “and/or” means one or all of the listed elements or a combination of any two or more of the listed elements.

Also herein, all numbers are assumed to be modified by the term “about” and preferably by the term “exactly.” As used herein in connection with a measured quantity, the term “about” refers to that variation in the measured quantity as would be expected by the skilled artisan making the measurement and exercising a level of care commensurate with the objective of the measurement and the precision of the measuring equipment used.

As used herein as a modifier to a property or attribute, the term “generally”, unless otherwise specifically defined, means that the property or attribute would be readily recognizable by a person of ordinary skill but without requiring absolute precision or a perfect match (e.g., within +/−20 % for quantifiable properties). The term “substantially”, unless otherwise specifically defined, means to a high degree of approximation (e.g., within +/−10% for quantifiable properties) but again without requiring absolute precision or a perfect match. Terms such as same, equal, uniform, constant, strictly, and the like, are understood to be within the usual tolerances or measuring error applicable to the particular circumstance rather than requiring absolute precision or a perfect match.

In a first aspect, a device is provided. The device comprises:
a flexible substrate including an adhesive surface on a first major side thereof;
an access component comprising a first electrically conductive component, wherein the first electrically conductive component is adhesively bonded to the adhesive surface of the flexible substrate, and a masking component that covers the first electrically conductive component opposite the adhesive surface of the flexible substrate;
a second electrically conductive component electrically connected to the first electrically conductive component and that extends from the first electrically conductive component on the first major side of the flexible substrate through to the second major side of the flexible substrate; and
an encapsulating layer that encapsulates the masking component and at least a portion of the first major side of the flexible substrate.

In a second aspect, a method of making a device is provided. The method comprises:
obtaining a flexible substrate having an adhesive surface on a first major side thereof and an access component, wherein the access component comprises a first electrically conductive component, wherein the first electrically conductive component is adhesively bonded to the adhesive surface of the flexible substrate, and a masking component that covers the first electrically conductive component opposite the adhesive surface of the flexible substrate;
providing a second electrically conductive component electrically connected to the first electrically conductive component and that extends from the first electrically conductive component on the first major side of the flexible substrate through to the second major side of the flexible substrate; and
applying an encapsulating layer that encapsulates the masking component and at least a portion of the first major side of the flexible substrate.

In a third aspect, a method of using a device is provided. The method comprises:
obtaining a device according to the first aspect described above in detail; and
removing at least a portion of the masking component from the device to expose the first electrically conductive component at the first major side of the flexible substrate.

The below disclosure relates to each of the first, second, and third aspects.

The present disclosure describes devices and methods for allowing access to an electronically conductive component which is embedded within a flexible electronic device. This enables the option of making a connection to the electrically conductive components and allows for post processing of the flexible electronic device.

It is often the case in the manufacture of electronic devices that sensitive components are protected with some type of encapsulant. In particular, in roll-to-roll assembly of such devices it may be advantageous to apply the encapsulant continuously over the entire surface of the devices, as this allows the practitioner to use traditional coating techniques such as roll coating and slot die coating. However, there are a variety of electrical sensors which require access to the environment (such as barometric pressure sensors, electrolytic sensors, chemical sensors, etc.), which may not function properly if they are fully covered. Therefore, there is a need for a technique to protect such devices during processing, while enabling access to the sensor (for example, through the removal of a masking layer) at a later point. Alternatively, this same approach can be used to enable access to a fully protected circuit at a later point, for example if one desires to attach a delicate component that cannot withstand the processing conditions required to build the device.

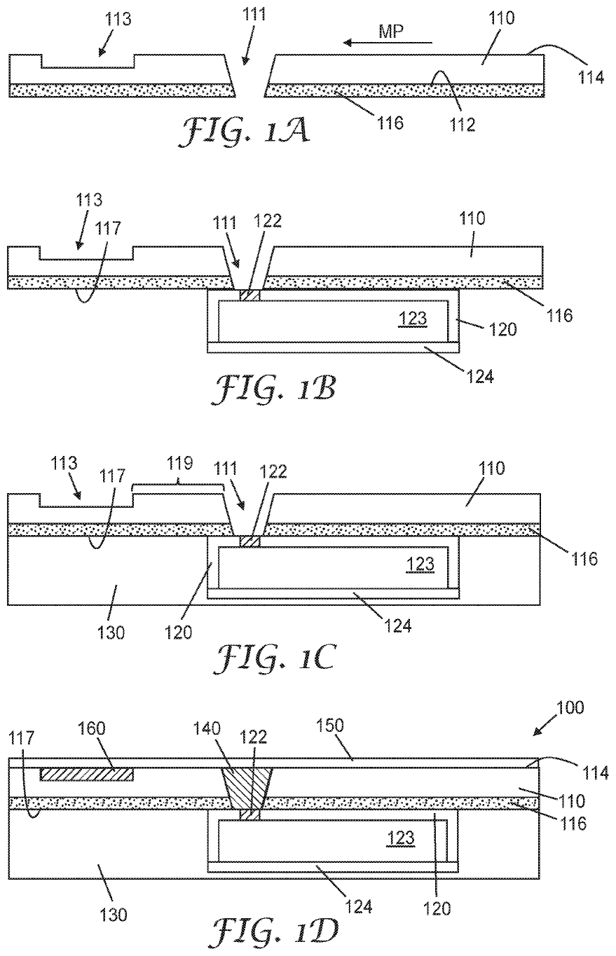

Referring to FIGS. 1A-1G, schematic cross-sectional views are provided of methods of making and/or using an exemplary device according to the present disclosure. For instance, in FIG. 1A, a portion of a device comprises a flexible substrate 110 that has a first major side 112 and a second opposing major side 114. The flexible substrate 110 extends along a major plane as indicated by an arrow MP in FIG. 1A. The flexible substrate 110 can include, for example, a polymer film such as a flexible polymer including, e.g., polyurethane, rubber, epoxy, acrylate, silicone, polyester, polyimide, polyethylene terephthalate (PET), polyethylene, polypropylene, polystyrene, silicone elastomer (e.g., PDMS), etc. It is to be understood in some embodiments, a portion of the flexible substrate 110 may be rigid, while the flexible substrate 110 as a whole can be flexible. The flexible substrate 110 may be elastic, having a modulus in the range, for example, between 0.1 MPa to 10 GPa. In some cases, the flexible substrate 110 is capable of being bent around a cylindrical object with a radius of curvature of up to 7.6 centimeters (cm) (3 inches), in some embodiments up to 6.4 cm (2.5 inches), 5 cm (2 inches), 3.8 cm (1.5 inch), or 2.5 cm (1 inch). In some cases, the flexible substrate 110 can be bent around a radius of curvature of at least 0.635 cm (¼ inch), 1.3 cm (½ inch) or 1.9 cm (¾ inch).

The flexible substrate 110 comprises an adhesive 116 on the first major side 112, which may be a single adhesive layer or a multilayer adhesive. The adhesive 116 provides an adhesive surface 117 of the flexible substrate 110.

In general, an adhesive material used herein can provide an adhesion strong enough such that the attached access component (including the first electrically conductive component) may not be easily displaced from an original position during subsequent handling. In some embodiments, the first electrically conductive component may be removed and/or repositioned without significantly damaging the flexible substrate. In some embodiments, the adhesive force may be high enough that the first electrically conductive component may not be easily removed or displaced without significantly damaging the flexible substrate. The adhesive material may also be capable of maintaining its structure, e.g., not reflowing into an adjacent through hole or microchannel.

Composition of the adhesive surface is not particularly limited as long as the first electrical component remains attached to the surface. Optionally, the adhesive surface comprises an adhesive including at least one of polyurethane, epoxy, silicone, urethane-acrylate, or (meth)acrylate. It is expressly contemplated that the adhesive may be a pressure-sensitive adhesive or a structural adhesive. In some embodiments, two-stage structural adhesives can be applied. For example, a first electrically conductive component such as a solid circuit die can be placed on a pressure-sensitive adhesive (PSA) which can be cured in a second step to form a structural adhesive. A suitable two-stage structural adhesive may experience a first curing step that initiates or catalyzes a reaction and enable some repositionability followed by a second curing step that finishes the reaction. In this case, “cure” means to cause a hardening or an increase in viscosity through a physical or chemical reaction such as, for example, by exposure to electromagnetic radiation or heating. In some embodiments, the adhering can be performed with, for example, a UV curable polyurethane compound. In some embodiments, a uniform layer of adhesive material can be provided onto the flexible substrate. In some embodiments, the adhesive material can be selectively applied onto the flexible substrate where a first electrically conductive component is to be attached.

Further, the flexible substrate 110 comprises a via 111 that is an opening defined by the flexible substrate 110, which passes from the second major side 114 through the first major side 112 and the adhesive 116. For instance, one suitable method for making a flexible substrate with vias (as well as attached components) is described in U.S. Application Publication No. 2022/0037278 (Mahajan et al.), which is incorporated herein by reference in its entirety. The depicted embodiment includes an optional microchannel 113 located on the second major side 114 of the flexible substrate 110. Optionally, a pattern of microchannels (not shown) is present on the second major side of the flexible substrate. One or more vias and microchannels may be formed on a flexible substrate by laser cutting, e.g., using a 400 watt Coherent E400i, CO2 laser, running at a 9.4 micron wavelength. For example, microchannels may be cut with one pass at a marking speed of 1000 mm/s with 100 kHz pulse rate, and about 64 watts of power. In some cases, microchannels formed in the flexible substrate are linear with a generally rectangular or hemispherical cross-section, cut to about ⅔ of the depth of the flexible substrate, and a width of about 160 micrometers. Vias (e.g., through holes) may be cut, for example, using a circular path at a marking speed of 1000 mm/sec, 100 kHz pulse rate, and about 28 watts of power with two passes, isolating an small section of the flexible substrate 110 and adhesive 116 which can be removed mechanically to fully open the via. Often, the via has a semi-cone shape with a top diameter of about 500 micrometers and a bottom diameter of about 300 micrometers.

Referring now to FIG. 1B, a portion of a device comprises a flexible substrate 110 according to the flexible substrate 110 depicted in FIG. 1A, but also comprising an access component 120 comprising a first electrically conductive component 122 and a masking component 124 that covers the first electrically conductive component 122 opposite the adhesive surface 117 of the flexible substrate 110. It is to be understood that in other embodiments, more than just the small area indicated to be the first electrically conductive component 122 is actually electrically conductive. For instance, the portion indicated as the access component 120 may also be electrically conductive, e.g., the access component may comprise an electrode such as an electrocardiogramcludes a metal and conductive gel (e.g., those ECG electrodes commercially available under the trade name “3M RED DOT ECG Electrodes” from 3M Company (St. Paul, MN). The access component optionally comprises a flexible material and/or a rigid material. The material for use in/as the access component is not particularly limited as long as an electrically conductive material is included somewhere that can be accessed behind the masking component. Nonlimiting exemplary materials include for instance, rubbers, plastics, metals, semiconductors, hydrogels, and carbon.

The first electrically conductive component is not particularly limited. In some embodiments, the first electrically conductive component 122 includes one or more ECG electrodes as mentioned above, and/or one or more solid circuit dies to attach to the adhesive surface 117 of the flexible substrate 110.

Each solid circuit die would have a major surface thereof being adhesively bonded to the adhesive surface 117 of the flexible substrate 110. Solid circuit dies typically each include one or more contact pads on their respective major surfaces. A solid circuit die can be aligned with respect to the flexible substrate 110 such that a contact pad at least partially overlies the corresponding via 111 (e.g., through hole) of the flexible substrate 110. A contact pad may have a smaller, roughly equivalent, or larger diameter than the via.

A solid circuit die employed in embodiments of the present disclosure can include one or more circuit chips having certain circuitry function(s). In some embodiments, a solid circuit die may include a circuit chip having one or more contact pads arranged along a surface thereof, a rigid semiconductor die, a printed circuit board (PCB), a flexible printed circuit (FPC), an ultra-thin chip, a radio frequency identification device (RFID), a near field communication (NFC) module, surface-mount devices, etc. In some embodiments a solid circuit die may include components such as LEDs, resistors, capacitors, switches, accelerometers, thermocouples, pressure sensors, light sensors, or any other appropriate component. In some embodiments, a solid circuit die may include batteries, such as coin cell batteries or thin film batteries, battery tabs, battery housings, or other connectors that may be used to provide power to the device. In some embodiments, a solid circuit die can be an ultra-thin chip with a thickness of about 2 micrometers to about 200 micrometers, about 5 micrometers to about 100 micrometers, or about 10 micrometers to about 100 micrometers. In some embodiments, a solid circuit die can include a rigid or flexible semiconductor die, e.g., a printed circuit board (PCB), or a flexible printed circuit (FPC). In some embodiments, a solid circuit die may include electrically conductive plates or traces. It is to be understood that the solid circuit dies described herein can include any suitable circuits to be disposed on a substrate. In some embodiments, one or more contact pads of a solid circuit die or the solid circuit die itself can be registered and connected to electrically conductive traces on a substrate.

As noted above, the access component 120 comprises a masking component 124 that covers the first electrically conductive component 122 opposite the adhesive surface 117 of the flexible substrate 110. Exemplary suitable materials for the masking component include for example and without limitation, a flexible polymeric film, an adhesive tape, a metal foil, an epoxy, a rubber, a paper substrate, or a rigid polymeric material.

Referring to FIG. 1C, a portion of a device comprises the flexible substrate 110 and the access component 120 depicted in FIG. 1B, but also comprising an encapsulating layer 130 that encapsulates the masking component 124 and at least a portion of the first major side 112 of the flexible substrate 110. In various embodiments, the encapsulating layer 130 is formed from a liquid encapsulant material that may include, for example, a dielectric material, a polymeric material, and the like. Examples of suitable liquid encapsulant materials include, for instance, polyurethane, epoxy, polythiolene, acrylates including urethane acrylates, silicones, silicone acrylates, and polydimethylsiloxane (PDMS). A liquid encapsulant material can flow around and cover the masking component 124, and other components (e.g., other portions of the access component 120), on the first major side 112 of the flexible substrate 110. In some embodiments, the liquid encapsulant material can be solidified by any suitable technique including, but not limited to, heating, application of radiation such as ultraviolet (UV), and combinations thereof. Once solidified, the liquid encapsulant material can form an encapsulating layer 130 as shown in FIG. 1C. It was unexpectedly discovered that application of liquid encapsulant did not flood the access component such that the first electrically conductive material got undesirably covered in encapsulant. Rather, it was possible to remove (e.g., peel back or make a hole) at least a portion of the masking component 124 underneath the encapsulating layer 130 and achieve access to the first electrically conductive material 122, for instance to electrically connect another component to the first electrically conductive material 122.

FIG. 1C further depicts an optional through hole 119 that connects the optional microchannel 113 to the via 111.

Referring to FIG. 1D, an exemplary device 100 is shown comprising the flexible substrate 110, the access component 120, and the encapsulating layer 130 depicted in FIG. 1C, but also comprising a second electrically conductive component 140 electrically connected to the first electrically conductive component 122. The second electrically conductive component 140 extends from the first electrically conductive component 122 through to the second major side 114 of the flexible substrate 110. In some cases, the second electrically conductive component 140 is provided using a conductive particle-containing liquid. For instance, a conductive particle-containing liquid may be flowed into the via 111 to make a direct contact to the first electrically conductive component 122. It is to be understood that in some embodiments, when a via (or through hole) is not fluidly-connected to a microchannel, an electrical contact can be directly formed to the first electrically conductive component by at least partially filling the via or through hole with an electrically conductive material. The conductive particle-containing liquid can be any electrically conductive liquid composition containing conductive particles that is flowable, or can be made to flow and can be any liquid composition that is electrically conductive in a liquid state (for example, metals), or is electrically non-conductive or weakly conductive in a liquid state and becomes electrically conductive when solidified.

The conductive particle-containing liquid includes an electrically conductive material, or an electrically non-conductive material that can be converted into an electrically conductive material, which is dispersed in a liquid to facilitate more uniform deposition by using, for example, a coater or sprayer.

The conductive particle-containing liquid can be deposited by various methods including, for example, chemical vapor deposition, physical vapor deposition, sputtering, spraying, air knife, gravure, dipping, kiss coating, flood coating, blading, immersion, Meyer rod, roll coating, slot die coating, inkjet printing, lithography, flexographic printing, screen printing, and mixtures and combinations thereof. Suitable electrically conductive materials for the conductive particle-containing liquid include, but are not limited to, metal particles, nanowires, metal salts that are conductive at room temperature or become conductive when heated or otherwise reduced to metals, conductive polymers, and mixtures and combinations thereof. In some embodiments, the conductive particle-containing liquid includes conductive inks including a conductive metal such as silver ink, silver nanoparticle ink, reactive silver ink, copper ink, and conductive polymer inks, as well as liquid metals or alloys (e.g., metals or alloys that melt at relatively low temperatures and solidify at room temperatures), and the like. In some embodiments, the conductive particle-containing liquid is a conductive ink that is activated or curable with actinic radiation such as, for example, a UV curable or activated ink.

In some embodiments, the conductive material in the conductive particle-containing liquid may be silver flakes or spheres, a blend of carbon/graphite particles or a blend of silver flakes/carbon particles. Particle sizes typically range from, for example, about 0.5 micrometers to about 10.0 micrometers in diameter. When these flakes or particles are suspended in the polymer binder, they are randomly spaced through the liquid. Once the solvent is evaporated, they condense, forming a conductive path or circuit. Of the conductive materials, silver is the least resistive and the most expensive while carbon/graphite offers the best combination of low resistance and low price. Suitable conductive inks are commercially available from, for example, Tekra, Inc., New Berlin, WI; Creative Materials, Inc., Ayer, MA; or NovaCentrix, Austin, TX.

Any non-corrosive liquid in which the conductive materials can form a stable dispersion can be used in the conductive particle-containing liquid, and suitable examples of liquid carriers include, but are not limited to, water, alcohols, ketones, ethers, hydrocarbons or an aromatic solvent (benzene, toluene, xylene, etc.). In some embodiments, the carrier liquid is volatile, having a boiling point of no more than 200 degrees C. (° C.), no more than 150° C., or no more than 100° C.

In addition, the conductive particle-containing liquid may contain additives or binders to control viscosity, corrosion, adhesion, and dispersion of the conductive material. Examples of suitable additives or binders include, but are not limited to, carboxy methyl cellulose (CMC), 2-hydroxy ethyl cellulose (HEC), hydroxy propyl methyl cellulose (HPMC), methyl cellulose (MC), poly vinyl alcohol (PVA), tripropylene glycol (TPG), and xanthan gum (XG), and surfactants such as ethoxy lates, alkoxylates, ethylene oxide and propylene oxide and their copolymers, sulfonates, sulfates, disulfonate salts, sulfosuccinates, phosphate esters, and fluorosurfactants (e.g., those available under the trade designation Zonyl from DowDuPont).

In one example, a conductive particle-containing liquid, or “ink”, includes, by weight, from 0.0025% to 0.1% surfactant (e.g., a preferred range is from 0.0025% to 0.05% for Zonyl FSO-100), from 0.02% to 4% viscosity modifier (e.g., a preferred range is 0.02% to 0.5% for HPMC), from 94.5% to 99.0% solvent and from 0.05% to 1.4% conductive materials. Representative examples of suitable surfactants include those available from DowDuPont, Wilmington, DE, under the trade designations Zonyl FSN, Zonyl FSO, and Zonyl FSH, those available from Millipore Sigma, St. Louis, MO, under the trade designations Triton (x100, x114, x45), those available from Evonik Industries, Parsippany, NJ, under the trade designations Dynol (604, 607), n-dodecyl b-D-maltoside and Novek. Examples of suitable viscosity modifiers include hydroxypropyl methyl cellulose (HPMC), methyl cellulose, xanthan gum, polyvinyl alcohol, carboxy methyl cellulose, hydroxy ethyl cellulose. Examples of suitable solvents that may be present includes the aforementioned liquid carriers, binders, or additives, including water and isopropanol. In another embodiment, the conductive particle-containing liquid 42 can include an adhesive such as, for example, adhesives dissolved in liquid solvents such as water acetone, toluene, methyl ethyl ketone (MEK), and the like.

The conductive particle-containing liquid can be cured, hardened or solidified by removing at least portion of the liquid carrier to leave a continuous layer of electrically conductive material that forms an electrically conductive trace, e.g., in the via 111 and/or in a microchannels 113. The conductive particle-containing liquid may be cured and/or hardened or sintered. “Cured or solidified” refers to a process where the solvent or liquid carrier is removed from the conductive particle-containing liquid to form an interconnect circuit pattern. Suitable curing conditions are well known in the art and include by way of example, heating, irradiating with visible or ultraviolet (UV) light, electron beams, and the like. Alternatively, “harden(s) or hardening” may be caused by solvent removal during drying, for example, without polymerization or cross-linking.

In the embodiments shown in at least FIGS. 1B-1D, the access component 120 defines a void 123 between the first electrically conductive component 122 and the masking component 124. A void 123 may be advantageous in providing space for manipulating the device when accessing the first electrically conductive component 122, e.g., to make electrical contact between the first electrically conductive component 122 and another electrically conductive component.

This embodiment includes an optional encapsulating layer 150. In such embodiments, the encapsulating layer 130 that encapsulates the masking component 124 and at least a portion of the first major side 117 of the flexible substrate 110 is a first encapsulating layer 130 and the device 100 further comprises a second encapsulating layer 150 that encapsulates the second electrically conductive component 140 and at least a portion of the second major side 114 of the flexible substrate 110.

Another optional feature that is shown in FIG. 1D is an electrically conductive channel trace 160 formed in the microchannel 113 of the flexible substrate 110. For instance, one or more electrically conductive channel traces may be formed by filling and/or coating the microchannel 113 with an electrically conductive material, such as described in detail above. A microchannel is configured with dimensions such as widths, depths, and lengths selected to allow a conductive particle-containing liquid placed in the microchannels to flow along the channel. The conductive particle-containing liquid may be placed in the microchannel by any suitable technique, and examples include, but are not limited to, chemical vapor deposition, physical vapor deposition, sputtering, spraying, air knife, gravure, dipping, kiss coating, flood coating, blading, immersion, Meyer rod, roll coating, slot die coating, inkjet printing, lithography, flexographic printing, screen printing, and mixtures and combinations thereof. In some embodiments, the dimensions of the microchannels are selected such that the conductive particle-containing liquid can be placed in the microchannels and flowed along the channels primarily by capillary force. In some embodiments, the conductive particle-containing liquid can be applied to the microchannels under pressure to enhance capillary flow, or can be moved through the microchannels by a pump, by application of a vacuum, and the like. In some embodiments, the conductive particle-containing liquid can be applied to the microchannels under pressure with an insignificant contribution from capillary flow, for example by roll coating the conductive particle-containing liquid.

Although FIGS. 1A-1D show one possible order of preparing a device according to the present disclosure, it is expressly contemplated that alternate orders of forming portions of the device are also suitable. For instance, although the masking component 124 is describe as a part of the access component 120, the masking component 124 may be attached to the device at a later time than the access component 120 is adhered to the adhesive surface 117 of the flexible substrate 110. In certain embodiments, obtaining a flexible substrate having an adhesive surface on a first major side and an access component including a first electrically conductive component involves receiving such a structure as assembled (e.g., from a vendor), whereas in other embodiments the obtaining involves performing a multi-step process to make such a structure (e.g., as described in detail in the Examples below). For instance, obtaining the flexible substrate having an adhesive surface on a first major side thereof and an access component may include covering the first electrically conductive component with the masking component opposite the adhesive surface of the flexible substrate.

In certain cases, providing the second electrically conductive component comprises providing a conductive particle-containing liquid in an aperture that extends from the first major side of the flexible substrate through to the second major side of the flexible substrate; and solidifying the conductive particle-containing liquid to form one or more electrically conductive channel traces to electrically connect to the first electrically conductive component.

An advantage of devices according to at least certain embodiments of the present disclosure is that the devices are useful for various different applications due to the ability to reveal the first electrically conductive component on demand, e.g., for attachment to any other desired electrically conductive component. Referring now to FIG. 1E, a device 100e has had its masking component removed. It is possible to partially or completely remove the masking component; in this depiction the masking component is fully removed and not present in the figures, to provide access to the first electrically conductive component 122.

It was unexpectedly discovered that it is possible to gain access to a first electrically conductive component attached to a flexible substrate and that has been masked and encapsulated, such as by removing at least a portion of the masking component from the device to expose the first electrically conductive component at the first major side of the flexible substrate. In some cases, removing at least a portion of the masking component 124 comprises forming a hole 125 in the device 100e through a full thickness of the first encapsulating layer and through a full thickness of the masking component. For example, the hole 125 may be as large in area as the access component 120, as shown in FIG. 1E, but it is expressly contemplated that the hole may be through only a portion of the area of the masking component and/or access component.

Referring to FIG. 1F, in some cases, removing at least a portion of the masking component 124 comprises forming a hole 125 in the device 100f through a full thickness of the first encapsulating layer that further includes forming a hole 127 through a full thickness of the flexible substrate 110. In this embodiment, the hole 127 extends through a full thickness of each of the access component 120, the adhesive 116, the flexible substrate 110, and the second encapsulating layer 150. Such a hole 127, as well as the hole 125, may be formed using a laser or punching out, for instance.

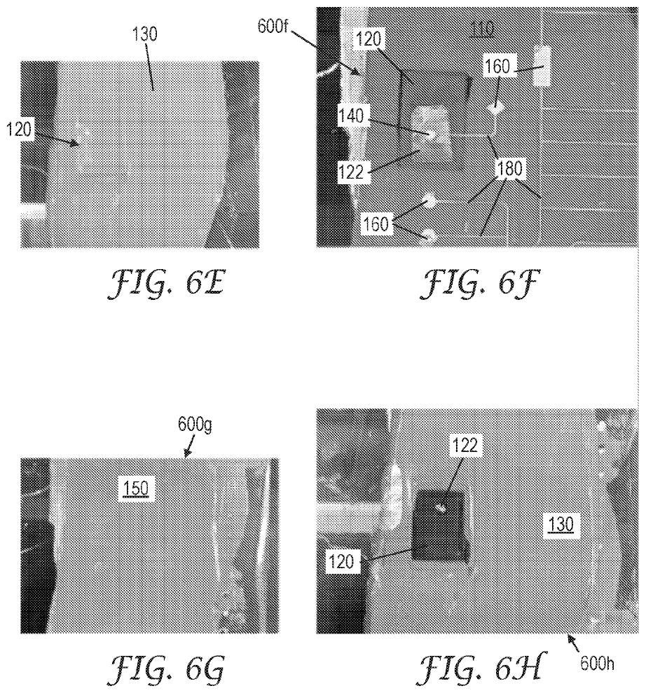

Referring to FIG. 5, in some cases removing at least a portion of the masking component 124 from a device 500 comprises peeling at least a portion of the masking component 124 apart from at least one of the first major side 112 of the flexible substrate 110 (shown) or the first electrically conductive component 122 (not shown).

Referring to FIG. 1G, in some embodiments, a method of making or using a device 100g further comprises attaching a third electrically conductive component 170 to the first electrically conductive component 122, e.g., opposite the second electrically conductive component 140. Often, the third electrically conductive component 170 is positioned adjacent a portion of the access component 120 to which the first electrically conductive component 122 is attached. The third electrically conductive component 170 is not particularly limited. In some cases, the third electrically conductive component 170 comprises a wire, for instance as shown in FIG. 1G. In other cases, the third electrically conductive component is a different type of component, such as a sensor. In this embodiment, the access component 122 includes a void 123 and at portion of the third electrically conductive component 170 is disposed within the void 123.

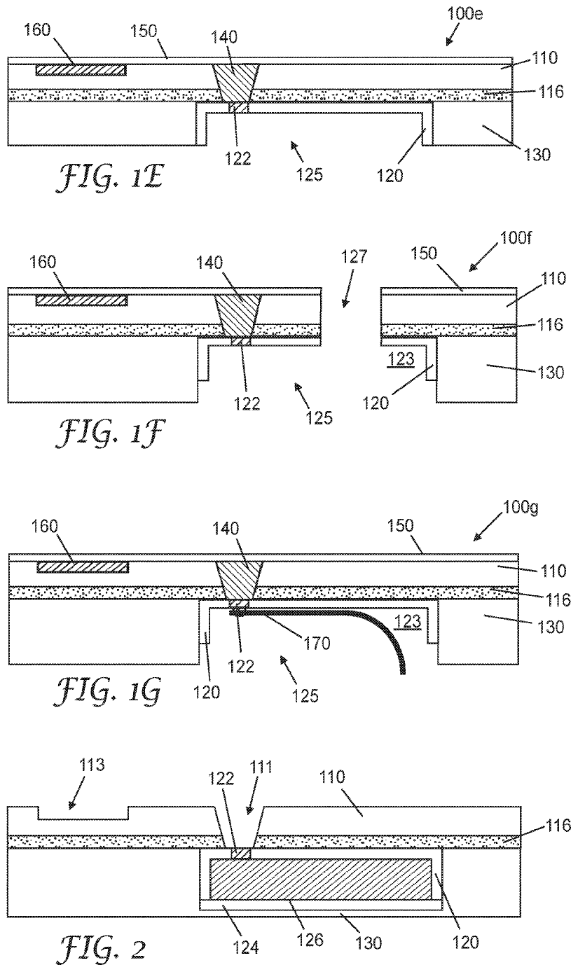

Referring to FIG. 2, in some cases, the access component 120 (e.g., of a portion of a device) defines a void (not shown, but similar to the void 123 in other figures) between the first electrically conductive component 122 and the masking component 124 and a method of making and/or using a device further comprises filling the void with an electrically conductive material 126. Alternatively, the access component 120 is sized to generally surround the first electrically conductive component 122 such there is no void between the interior of the access component 120 and the first electrically conductive component 122.

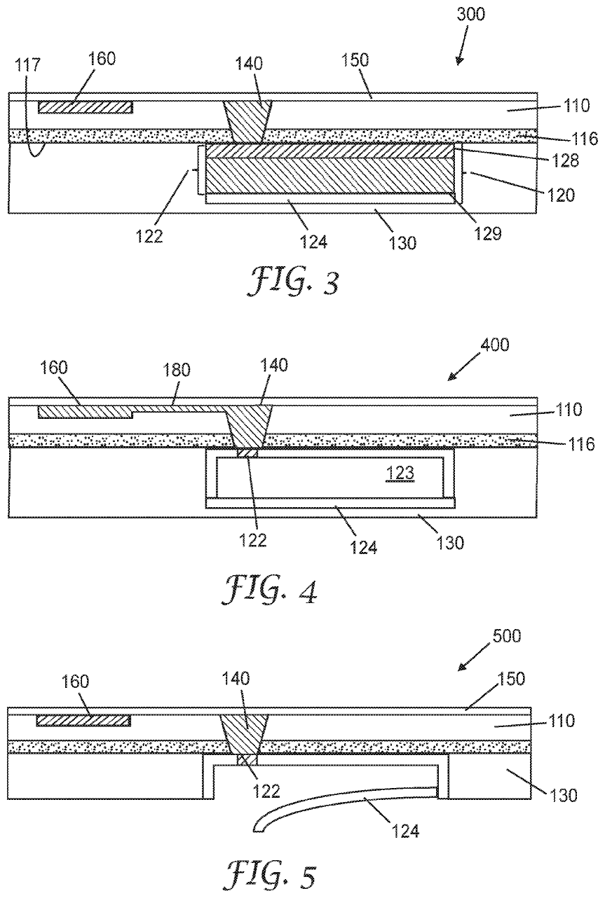

Referring to FIG. 3, in some cases, the first electrically conductive component 122 of a device 300 extends from the first major side 117 of the flexible substrate 110 through to the masking component 124. In one select embodiment, the first electrically conductive component 122 comprises an ECG electrode comprising a metal component 128 and an electrically conductive gel 129. Often, an ECG electrode further comprises a liner that can be removed to expose the electrically conductive gel 129, and the liner can act as the masking component 124, but alternatively other masking components could be used with an ECG electrode.

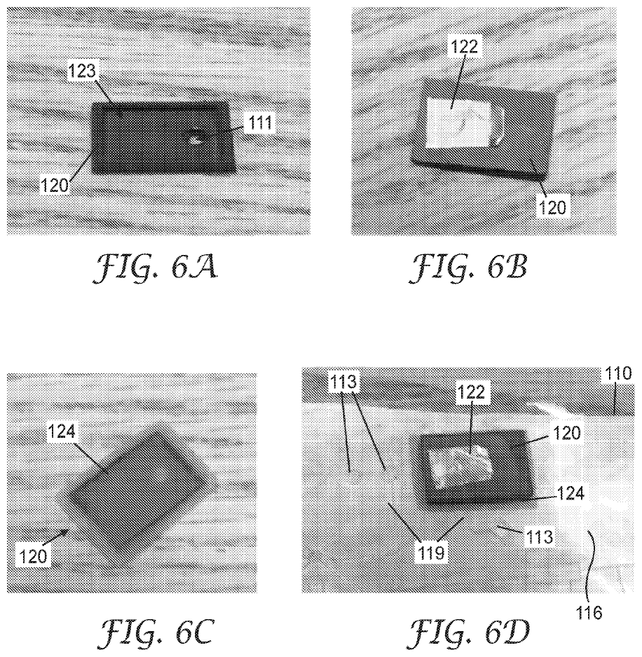

Referring to FIG. 4, in some cases, a device 400 further comprises one or more through holes 119 (as shown in FIG. 1C) containing electrically conductive material 180 and connected to the pattern of microchannels 160. At least one of the through holes 119 containing electrically conductive material 180 extends through the flexible substrate 110 between the microchannel 113/electrically conductive channel trace 160 and the via 111/second electrically conductive component 140) along the second major side 114. One or more electrically conductive channel traces 160 and 180 are formed in the pattern of microchannels 113 and the through holes 119 to electrically connect to the first electrically conductive component 122. In some cases, a circuit further includes one or more other components, e.g., a chip, a sensor, a battery, etc. For instance, suitable electrical components include a battery such as a coin cell battery as described in U.S. Application Publication No. 2022/0037278 (Mahajan et al.).

### Exemplary Embodiments

In a first embodiment, the present disclosure provides a device. The device comprises a flexible substrate including an adhesive surface on a first major side thereof and an access component that comprises a first electrically conductive component and a masking component that covers the first electrically conductive component opposite the adhesive surface of the flexible substrate. The first electrically conductive component is adhesively bonded to the adhesive surface of the flexible substrate. The device also comprises a second electrically conductive component electrically connected to the first electrically conductive component and that extends from the first electrically conductive component on the first major side of the flexible substrate through to the second major side of the flexible substrate. Further, the device comprises an encapsulating layer that encapsulates the masking component and at least a portion of the first major side of the flexible substrate.

In a second embodiment, the present disclosure provides a device according to the first embodiment, wherein the first electrically conductive component comprises a solid circuit die.

In a third embodiment, the present disclosure provides a device according to the second embodiment, wherein the second electrically conductive component extends from the second major side of the flexible substrate through to the masking component.

In a fourth embodiment, the present disclosure provides a device according to the first embodiment or the second embodiment, wherein the access component defines a void between the first electrically conductive component and the masking component.

In a fifth embodiment, the present disclosure provides a device according to any of the first through fourth embodiments, wherein the access component comprises a flexible material.

In a sixth embodiment, the present disclosure provides a device according to any of the first through fourth embodiments, wherein the access component comprises a rigid material.

In a seventh embodiment, the present disclosure provides a device according to any of the first through sixth embodiments, wherein the masking component comprises a flexible polymeric film, an adhesive tape, a metal foil, an epoxy, a rubber, a paper substrate, or a rigid polymeric material.

In an eighth embodiment, the present disclosure provides a device according to any of the first through seventh embodiments, wherein the encapsulating layer that encapsulates the masking component and at least a portion of the first major side of the flexible substrate is a first encapsulating layer and the device further comprises a second encapsulating layer that encapsulates the second electrically conductive component and at least a portion of the second major side of the flexible substrate.

In a ninth embodiment, the present disclosure provides a device according to any of the first through eighth embodiments, wherein the flexible substrate further comprises a pattern of microchannels on a second major side opposite the first major side.

In a tenth embodiment, the present disclosure provides a device according to the ninth embodiment, further comprising one or more through holes connected to the pattern of microchannels, at least one of the through holes extending through the flexible substrate between the first and second major sides.

In an eleventh embodiment, the present disclosure provides a device according to the tenth embodiment, further comprising one or more electrically conductive channel traces formed in the pattern of microchannels and the through holes to electrically connect to the first electrically conductive component.

In a twelfth embodiment, the present disclosure provides a device according to any of the first through eleventh embodiments, wherein the adhesive surface comprises an adhesive including at least one of polyurethane, epoxy, silicone, urethane-acrylate, or (meth)acrylate.

In a thirteenth embodiment, the present disclosure provides a method of making a device. the method comprises obtaining a flexible substrate including an adhesive surface on a first major side thereof and an access component that includes a first electrically conductive component and a masking component that covers the first electrically conductive component opposite the adhesive surface of the flexible substrate. The first electrically conductive component is adhesively bonded to the adhesive surface of the flexible substrate. The method also includes providing a second electrically conductive component electrically connected to the first electrically conductive component and that extends from the first electrically conductive component on the first major side of the flexible substrate through to the second major side of the flexible substrate. Further, the method includes applying an encapsulating layer that encapsulates the masking component and at least a portion of the first major side of the flexible substrate.

In a fourteenth embodiment, the present disclosure provides a method of making a device according to the thirteenth embodiment, wherein providing the second electrically conductive component comprises providing a conductive particle-containing liquid in an aperture that extends from the first major side of the flexible substrate through to the second major side of the flexible substrate, and solidifying the conductive particle-containing liquid to form one or more electrically conductive channel traces to electrically connect to the first electrically conductive component.

In a fifteenth embodiment, the present disclosure provides a method of making a device according to the thirteenth embodiment or the fourteenth embodiment, wherein obtaining the flexible substrate having an adhesive surface on a first major side thereof and an access component comprises covering the first electrically conductive component with the masking component opposite the adhesive surface of the flexible substrate.

In a sixteenth embodiment, the present disclosure provides a method of making a device according to any of the thirteenth through fifteenth embodiments, wherein the access component defines a void between the first electrically conductive component and the masking component electrically conductive component.

In a seventeenth embodiment, the present disclosure provides a method of making a device according to the sixteenth embodiment, wherein the access component comprises a flexible material.

In an eighteenth embodiment, the present disclosure provides a method of making a device according to any of the thirteenth through seventeenth embodiments, wherein the encapsulating layer that encapsulates the cap and at least a portion of the first major side of the flexible substrate is a first encapsulating layer and the method further comprises applying a second encapsulating layer that encapsulates the second electrically conductive component and at least a portion of the second major side of the flexible substrate.

In a nineteenth embodiment, the present disclosure provides a method of making a device according to any of the thirteenth through eighteenth embodiments, further comprising removing at least a portion of the masking component from the device to expose the first electrically conductive component at the first major side of the flexible substrate.

In a twentieth embodiment, the present disclosure provides a method of making a device according to the nineteenth embodiment, wherein removing at least a portion of the masking component comprises forming a hole in the device through a full thickness of the first encapsulating layer and through a full thickness of the masking component.

In a twenty-first embodiment, the present disclosure provides a method of making a device according to the nineteenth embodiment or the twentieth embodiment, wherein removing at least a portion of the masking component comprises peeling at least a portion of the masking component apart from at least one of the first major side of the flexible substrate or the first electrically conductive component.

In a twenty-second embodiment, the present disclosure provides a method of making a device according to any of the nineteenth through twenty-first embodiments, further comprising attaching a third electrically conductive component to the first electrically conductive component.

In a twenty-third embodiment, the present disclosure provides a method of using a device. The method comprises obtaining a device of any of the first through twelfth embodiments and removing at least a portion of the masking component from the device to expose the first electrically conductive component at the first major side of the flexible substrate.

In a twenty-fourth embodiment, the present disclosure provides a method of using a device according to the twenty-third embodiment, wherein removing at least a portion of the masking component comprises forming a hole in the device through a full thickness of the first encapsulating layer and through a full thickness of the masking component.

In a twenty-fifth embodiment, the present disclosure provides a method of using a device according to the twenty-third embodiment or the twenty-fourth embodiment, wherein removing at least a portion of the masking component comprises peeling at least a portion of the masking component apart from at least one of the first major side of the flexible substrate or the first electrically conductive component.

In a twenty-sixth embodiment, the present disclosure provides a method of using a device according to any of the twenty-third through twenty-fifth embodiments, further comprising attaching a third electrically conductive component to the first electrically conductive component.

In a twenty-sixth embodiment, the present disclosure provides a method of using a device according to any of the twenty-third through twenty-sixth embodiments, wherein the access component defines a void between the first electrically conductive component and the masking component and wherein the method further comprising filling the void with an electrically conductive material.

### EXAMPLES

Objects and advantages of this disclosure are further illustrated by the following examples, but the particular materials and amounts thereof recited in these examples, as well as other conditions and details, should not be construed to unduly limit this disclosure. Unless otherwise noted or otherwise apparent from the context, all parts, percentages, ratios, etc. in the Examples and the rest of the specification are by weight.

### Example 1

The flexible substrate of Example 1 was prepared using a stack with a layered construction of: 3M silicone adhesive transfer tape 91022 (2 mils or 50.8 micrometers thick, obtained under trade designation “3M ADHESIVE TRANSFER TAPE 91022”, from 3M Company, St. Paul, MN)/3M COTRAN polyurethane (3.5 mils or 88.9 micrometers thick, obtained under trade designation “3M COTRAN ETHYLENE VINYL ACETATE MEMBRANE FILM”, from 3M Company, St. Paul, MN)/1.5 mil or 38.1 micrometers thick PET liner. The layered construction was drilled with a laser so that the laser cut through the PET liner into the polyurethane to form a pattern of microchannels and cut completely through all the layers to form through holes.

The laser used to make the microchannels and through holes was a 400 watt Coherent E400i, CO2 laser, running at a 9.4 micron wavelength (obtained from Coherent, Inc., Santa Clara, CA). The laser was directed at the PET side of the layered construction. The partial channels were cut with one pass at marking speed of 1000 mm/s with 100 kHz pulse rate, and about 64 watts of power. The microchannels formed in the substrate were substantially linear with a generally rectangular or hemispherical in cross-section, cut into about ⅔ of the polyurethane layer, and a width of about 160 micrometers. The through holes were cut using a circular path at a marking speed of 1000 mm/sec, 100 kHz pulse rate, and about 28 watts of power with two passes. The through holes formed were semi-cone shaped with a top diameter of about 500 micrometers and a bottom diameter of about 300 micrometers.

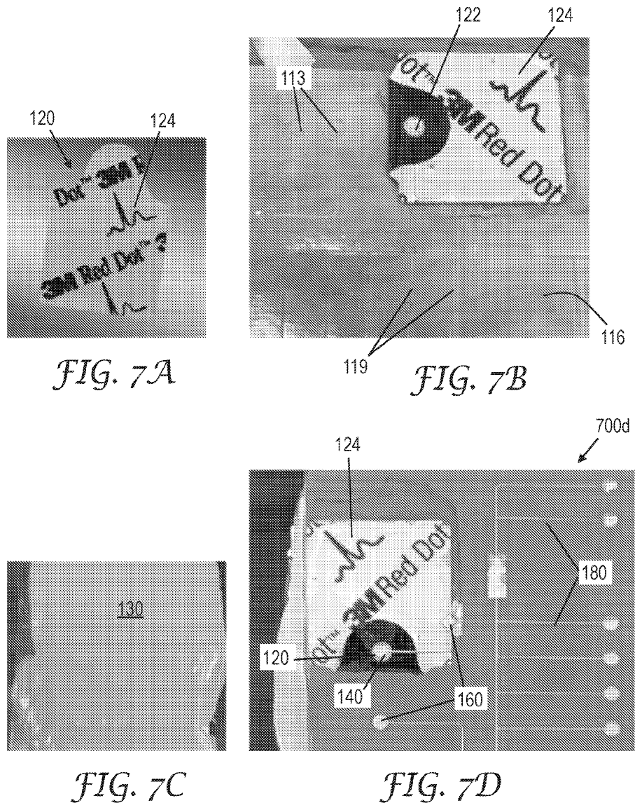

Referring to FIG. 6A, an access component 120 was made by laser etching a piece of 3M Bumpon resilient rollstock SJ5615 (1.59 mm thick, obtained under trade designation “3M BUMPON RESILIENT ROLLSTOCK SJ5615”, from 3M Company, St. Paul, MN) rubber so that a cavity (e.g., void) 123 and through hole (e.g., via) 111 were formed using a Universal Laser Systems 75 watt CO2 laser VLS 6.60 (obtained from Universal laser Systems, Inc., Scottsdale, AZ). The center cavity was raster cut at 20% speed 100% power, and the through hole and the edges were vector cut all the way through at 5% speed 100% power. Referring to FIG. 6B, Z-access conductive copper tape (obtained under trade designation “3M CONDUCTIVE COPPER FOIL TAPE 3313” from 3M Company, ST. Paul, MN) as a first electrically conductive component 122 was placed over the through hole (111) on the non-cavity side of the component 120 to form contact pads. Referring to FIGS. 6C, 3M Polyester film tape 850 (obtained under trade designation “3M POLYESTER FILM TAPE 850” from 3M Company, St. Paul, MN) was placed on the cavity side of the access component 120 as a masking component 124.

Referring to FIG. 6D, the access component 120 was placed directly on the adhesive surface 116 of the film stack (e.g., flexible substrate) 110, with the first electrically conductive component 122 (e.g., contact pads) facing down, and then pressed with a force for a few seconds to form a strong adhesive bonding. The microchannels 119 and through holes 113 of the flexible substrate 110 were arranged to form contacts to the configuration of the contact pads of the first electrically conductive component 122 on the access component 120.

Referring to FIG. 6E, an encapsulating layer 130 of 3M ESPE EXPRESS Imprint VPS Impression Material Light Body Regular Set 7302 (obtained under trade designation “3M ESPE EXPRESS Imprint VPS Impression Material, 7300 Series” from 3M Company, St. Paul, MN) was applied over the adhesive 116 and the access component 120 such that the tallest component was encapsulated under a thin layer of encapsulant. This was done by coating the encapsulating material over the top of the access component 120 using an adjustable notch bar adjusted to just touch the top of the component then moved up 5 mils (127 micrometers). Then a larger than needed quantity of encapsulant was applied to the edge of the device and the notch bar was run across the flexible substrate (110) to coat over the access component 120 and adhesive (116). This encapsulating layer 130 was cured at room temperature for 1 hour.

Referring to FIG. 6F, a silver flake ink available under the trade designation “127-07” from Creative Materials, Ayer, MA. A 40% silver loading was then doctor bladed in the pattern of microchannels and through holes to make contact to the contact pads of the first electrically conductive component 122 in the through holes. The silver ink was solidified by heating at 98° C. for about 5 to 10 minutes to form a second electrically conductive component 140, forming a device 600f, which also includes electrically conductive traces 180 and microchannels 160. The PET liner was removed from the flexible substrate 110 after the filling of the silver ink.

Referring to FIG. 6G, another encapsulating layer 150 of 3M ESPE EXPRESS Imprint VPS Impression Material Light Body Regular Set 7302 was coated over the silver ink and the polyurethane side of the device 600g, having a thickness of approximately 2 mils (50.8 micrometers). This was coated using the same method as above by setting a notch bar to the thickness of the device 600g, then increasing the gap by 5 mils (127 micrometers), applying an amount of encapsulant, then running the device under the notch bar. This second encapsulating layer 150 was then allowed to cure for 1 hour at room temperature.

Referring to FIG. 6H, the masking component has been removed from the access component 120 by taking a tweezer and grabbing the visible (bulge under encapsulant) corner of the masking component (not shown) and pulling to expose the first electrically conductive component 122.

### Example 2

The flexible substrate of Example 2 was prepared using a stack with a layered construction of:

3M silicone adhesive transfer tape 91022 (2 mils or 50.8 micrometers thick, obtained under trade designation “3M ADHESIVE TRANSFER TAPE 91022”, from 3M Company, St. Paul, MN)/3M COTRAN polyurethane (3.5 mils or 88.9 micrometers thick, obtained under trade designation “3M COTRAN ETHYLENE VINYL ACETATE MEMBRANE FILM”, from 3M Company, St. Paul, MN))/1.5 mil or 38.1 micrometers thick PET liner. The layered construction was drilled with a laser so that the laser cut through the PET liner into the polyurethane to form a pattern of microchannels and cut completely through all the layers to form through holes.

The laser used to make the microchannels and through holes was a 400 watt Coherent E400i, CO2 laser, running at a 9.4 micron wavelength (obtained from Coherent, Inc., Santa Clara, CA). The laser was directed at the PET side of the layered construction. The partial channels were cut with one pass at marking speed of 1000 mm/s with 100 kHz pulse rate, and about 64 watts of power. The microchannels formed in the substrate were substantially linear with a generally rectangular or hemispherical in cross-section, cut into about ⅔ of the polyurethane layer, and a width of about 160 micrometers. The through holes were cut using a circular path at a marking speed of 1000 mm/sec, 100 kHz pulse rate, and about 28 watts of power with two passes. The through holes formed were semi-cone shaped with a top diameter of about 500 micrometers and a bottom diameter of about 300 micrometers.

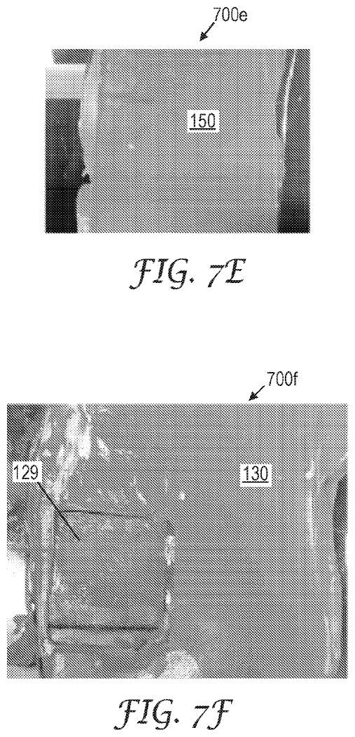

Referring to FIG. 7A, an access component 120 was a 3M Red Dot ECG electrode 2360 (obtained under trade designation “3M RED DOT ECG ELECTRODE 2360” from 3M Company, St. Paul, MN). The ECG electrode has a contact pad that was folded over so that it faced away from the electrode itself, and a liner on the electrode side that was the masking component 124.

Referring to FIG. 7B, the access component 120 was placed directly on the adhesive surface 116 of the flexible substrate 110, with the contact pad, as a first electrically conductive component 122, facing down, and then pressed with a force for a few seconds to form a strong adhesive bonding. The microchannels 119 and through holes 113 were arranged to form contacts to the configuration of the contact pad 122 on the access component 124.

Referring to FIG. 7C, an encapsulating layer 130 of 3M ESPE EXPRESS Imprint VPS Impression Material Light Body Regular Set 7302 was applied over the adhesive and the access component such that the tallest component was encapsulated under a thin layer of encapsulant. This was done by coating the encapsulating material over the top of the component using an adjustable notch bar adjusted to just touch the top of the component then moved up 5 mils (127 micrometers). Then a larger than needed quantity of encapsulant was applied to the edge of the device and the notch bar was run across the film to coat over the component and adhesive. This encapsulating layer 130 was cured at room temperature for 1 hour.

Referring to FIG. 7D, a silver flake ink was available under the trade designation “127-07” from Creative Materials, Ayer, MA. A 40% silver loading was then doctor bladed in the pattern of microchannels and through holes to make contact to the contact pads 122 of the ECG electrode in the through holes. The silver ink was solidified by heating at 98° C. for about 5 to 10 minutes to form a second electrically conductive component 140, forming a device 700d, which also includes electrically conductive traces 180 and microchannels 160. The PET liner was removed from the flexible substrate 110 after the filling of the silver ink.

Referring to FIG. 7E, another encapsulating layer 150 of 3M ESPE EXPRESS Imprint VPS Impression Material Light Body Regular Set 7302 was coated over the silver ink and the polyurethane side of the device 700e, having a thickness of approximately 2 mils (50.8 micrometers). This was coated using the same method as above by setting a notch bar to the thickness of the device, then increasing the gap by 5 mils (127 micrometers), applying an amount of encapsulant, then running the device under the notch bar. This second encapsulating layer 150 was then allowed to cure for 1 hour at room temperature.

Referring to FIG. 7F, the masking component (not shown) was removed from the access component of the device 700f by taking a tweezer and grabbing the visible (bulge under encapsulant) corner of the masking component and pulling to expose the first electrically conductive component, which in this case was electrically conductive gel 129 of the ECG electrode.

All of the patents and patent applications mentioned above are hereby expressly incorporated by reference. The embodiments described above are illustrative of the present invention and other constructions are also possible. Accordingly, the present invention should not be deemed limited to the embodiments described in detail above and shown in the accompanying drawings, but instead only by a fair scope of the claims that follow along with their equivalents.

1. A device comprising:
a flexible substrate including an adhesive surface on a first major side thereof;
an access component comprising a first electrically conductive component, wherein the first electrically conductive component is adhesively bonded to the adhesive surface of the flexible substrate, and a masking component that covers the first electrically conductive component opposite the adhesive surface of the flexible substrate;
a second electrically conductive component electrically connected to the first electrically conductive component and that extends from the first electrically conductive component on the first major side of the flexible substrate through to the second major side of the flexible substrate; and
an encapsulating layer that encapsulates the masking component and at least a portion of the first major side of the flexible substrate.

2. The device of claim 1, wherein the first electrically conductive component comprises a solid circuit die.

3. The device of claim 1, wherein the access component defines a void between the first electrically conductive component and the masking component.

4. The device of claim 1, wherein the masking component comprises a flexible polymeric film, an adhesive tape, a metal foil, an epoxy, a rubber, a paper substrate, or a rigid polymeric material.

5. The device of claim 1, wherein the encapsulating layer that encapsulates the masking component and at least a portion of the first major side of the flexible substrate is a first encapsulating layer and the device further comprises a second encapsulating layer that encapsulates the second electrically conductive component and at least a portion of the second major side of the flexible substrate.

6. The device of claim 1, wherein the flexible substrate further comprises a pattern of microchannels on a second major side opposite the first major side.

7. The device of claim 6, further comprising one or more through holes connected to the pattern of microchannels, at least one of the through holes extending through the flexible substrate between the first and second major sides.

8. The device of claim 7, further comprising one or more electrically conductive channel traces formed in the pattern of microchannels and the through holes to electrically connect to the first electrically conductive component.

9. A method of making a device comprising:
obtaining a flexible substrate having an adhesive surface on a first major side thereof and an access component, wherein the access component comprises a first electrically conductive component, wherein the first electrically conductive component is adhesively bonded to the adhesive surface of the flexible substrate, and a masking component that covers the first electrically conductive component opposite the adhesive surface of the flexible substrate;
providing a second electrically conductive component electrically connected to the first electrically conductive component and that extends from the first electrically conductive component on the first major side of the flexible substrate through to the second major side of the flexible substrate; and
applying an encapsulating layer that encapsulates the masking component and at least a portion of the first major side of the flexible substrate.

10. The method of claim 9, wherein providing the second electrically conductive component comprises:
providing a conductive particle-containing liquid in an aperture that extends from the first major side of the flexible substrate through to the second major side of the flexible substrate; and
solidifying the conductive particle-containing liquid to form one or more electrically conductive channel traces to electrically connect to the first electrically conductive component.

11. The method of claim 9, wherein obtaining the flexible substrate having an adhesive surface on a first major side thereof and an access component comprises covering the first electrically conductive component with the masking component opposite the adhesive surface of the flexible substrate.

12. The method of claim 9, wherein the access component defines a void between the first electrically conductive component and the masking component electrically conductive component.

13. The method of claim 9, further comprising removing at least a portion of the masking component from the device to expose the first electrically conductive component at the first major side of the flexible substrate.

14. The method of claim 13, further comprising attaching a third electrically conductive component to the first electrically conductive component.

15. A method of using a device, the method comprising:
obtaining a device of claim 1; and
removing at least a portion of the masking component from the device to expose the first electrically conductive component at the first major side of the flexible substrate.

16. The method of claim 15, wherein removing at least a portion of the masking component comprises forming a hole in the device through a full thickness of the first encapsulating layer and through a full thickness of the masking component.

17. The method of claim 15, wherein removing at least a portion of the masking component comprises peeling at least a portion of the masking component apart from at least one of the first major side of the flexible substrate or the first electrically conductive component.

18. The method of claim 15, further comprising attaching a third electrically conductive component to the first electrically conductive component.

19. The method of claim 15, wherein the access component defines a void between the first electrically conductive component and the masking component and wherein the method further comprising filling the void with an electrically conductive material.
